(19) United States
(12) Patent Application Publication
(10) Pub. No.: US 2022/0062165 A1
(43) Pub. Date: Mar. 3, 2022

(54) COMPOSITIONS AND METHODS FOR INTRANASAL DELIVERY OF PREGNENOLONE

(71) Applicant: M et P Pharma AG, Emmetten (CH)

(72) Inventor: Claudia Mattern, Emmetten (CH)

(73) Assignee: M et P Pharma AG, Emmetten (CH)

(21) Appl. No.: 17/047,542

(22) PCT Filed: Apr. 16, 2019
(86) PCT No.: PCT/IB2019/053132

§ 371 Date: Oct. 14, 2020

Related U.S. Application Data
(60) Provisional application No. 62/658,946, filed on Apr. 17, 2018.

(51) Int. Cl. A61K 9/00; A61K 31/573; A61K 9/06; A61P 25/00

(52) U.S. Cl. CPC A61K 9/0043; A61P 25/00; A61K 9/06; A61K 31/573

(57) ABSTRACT

This invention relates to methods of increasing activity of the neurotransmitter acetylcholine in specific brain regions to treat diseases or disorders associated with reduced acetylcholine activity. In particular, the methods relate to intranasal administration of pregnenolone in only one nostril increasing acetylcholine activity only in the amygdala corresponding to this nostril, thus, providing ipsilateral increase of acetylcholine activity.

### CROSS-REFERENCE TO RELATED APPLICATIONS

This application claims priority to U.S. Provisional Application 62/658,946 filed Apr. 17, 2018, the entire contents of which are incorporated herein by reference.

### FIELD

Described herein are compositions and methods intranasal delivery of pregnenolone, useful, for example, for increasing acetylcholine activity in specific brain regions.

### BACKGROUND

Neurosteroids and neurotransmitters are compounds active in the brain that have specific roles in regulating normal brain function, including regulating cognition, feeding, emotion, motivation, and motor skills. See Zheng, P., “Neuroactive steroid regulation of neurotransmitter release in the CNS: action, mechanism and possible significance,” Prog. Neurobiol., 89, 134-152 (2009). Abnormal neurosteroid and neurotransmitter function and/or concentration is associated with numerous central nervous system (CNS) disorders, such as Schizophrenia, stroke, depression, Parkinson's and Alzheimers' disease. The neurosteroid pregnenolone increases acetylcholine (Ach) release in the brain. Acetylcholine is a prominent neurotransmitter of the cholinergic transmission system, and increased acetylcholine release in the amygdala is essential for memory processing and learning. The brain contains a number of cholinergic areas, each with distinct functions. They play an important role in arousal, attention, memory and motivation. See Hasselmo, M. E., “The role of acetylcholine in learning and memory,” Curr. Opin. Neurobiol., 16, 710-715 (2006). Acetylcholine activity is essential for healthy cognitive functions, and evidence suggests that both concentration and function of acetylcholine is impaired in Alzheimer's disease patients, making acetylcholine a key target for treating Alzheimer's disease. See Francis, P. T., “The interplay of neurotransmitters in Alzheimer's disease,” CNS Spectr., 10, 6-9 (2005). Currently, the main strategy for increasing acetylcholine activity in the brain of a patient suffering from decreased acetylcholine transmission is to administer acetylcholinesterase inhibitors, but their applicability is limited due to their toxicity. See Colovic M. B. et al., “Acetylcholinesterase inhibitors: Pharmacology and Toxicology,” Current Neuropharmacology 11, 315-335 (2013).

There is a need therefore for compositions and methods for increasing acetylcholine activity in specific regions of the brain.

### SUMMARY

Described herein are methods of ipsilaterally increasing acetylcholine activity in brain tissue of a subject in need thereof, particularly a non-rodent subject, comprising intranasally administering to the subject a pregnenolone formulation, wherein the pregnenolone formulation is a pharmaceutical composition adapted for intranasal administration comprising an effective amount of pregnenolone in a pharmaceutically acceptable carrier. The subject may be a human, a non-human primate, a dog, a cat, a cow, a sheep, a horse, or a rabbit.

In some embodiments, the pregnenolone formulation is administered only to one nostril, and acetylcholine activity is increased in an ipsilateral brain hemisphere of said nostril; in some embodiments, acetylcholine activity is not substantially increased in a contralateral brain hemisphere of said nostril.

In some embodiments, the method results in increased acetylcholine activity in amygdala of the subject. In some embodiments, the method results in increased acetylcholine activity in hippocampus of the subject.

In some embodiments, the acetylcholine activity is increased within 10 minutes. In some embodiments, acetylcholine activity in the brain tissue is sustained for at least 60 minutes, or for at least 100 minutes.

In some embodiments, the effective amount of pregnenolone is from about 0.01 mg to about 2.0 mg per kilogram of bodyweight of the subject.

In some embodiments, the pharmaceutically acceptable carrier comprises (a) at least one lipophilic or partly lipophilic carrier present in an amount of from about 60% to about 98% by weight of the formulation; (b) at least one compound having surface tension decreasing activity present in an amount of from about 1% to about 20% by weight of the formulation; and (c) at least one viscosity regulating agent present in an amount of from about 0.5% to about 10% by weight of the formulation.

In some embodiments, the pregnenolone is loaded onto a surface of a porous excipient located inside pores of the porous excipient.

In some embodiments, the subject is suffering from a disease or condition associated with decreased acetylcholine activity in the brain, such as schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, or sleep disorders.

In some embodiments, the method is effective to improve cognitive function such as memory and learning deficits.

Also provided are pregnenolone formulations for use in ipsilaterally increasing acetylcholine activity in brain tissue of a subject in need thereof, particularly a non-rodent subject, or for use in treating a disease or condition in a subject in need thereof selected from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders, wherein the pregnenolone formulations are pharmaceutical compositions adapted for intranasal administration comprising an effective amount of pregnenolone in a pharmaceutically acceptable carrier. In some embodiments, the pregnenolone formulation is adapted for intranasal administration to only one nostril of the subject. In some embodiments, the pregnenolone formulation is administered only to one nostril, and acetylcholine activity is increased in an ipsilateral brain hemisphere of said nostril. In some embodiments, acetylcholine activity is not substantially increased in a contralateral brain hemisphere of said nostril. In some embodiments, the use additionally or alternatively results in increased acetylcholine activity in amygdala of the subject. In some embodiments, the use additionally or alternatively results in increased acetylcholine activity in hippocampus of the subject. In some embodiments, the acetylcholine activity is increased within 10 minutes. In some embodiments, acetylcholine activity in the brain tissue is sustained for at least 60 minutes. In some embodiments, acetylcholine activity in the brain tissue is sustained for at least 100 minutes. In some embodiments, the effective amount of pregnenolone is from about 0.01 mg to about 2.0 mg per kilogram of bodyweight of the subject. In any embodiments, the pharmaceutically acceptable carrier may comprise (a) at least one lipophilic or partly lipophilic carrier present in an amount of from about 60% to about 98% by weight of the formulation; (b) at least one compound having surface tension decreasing activity present in an amount of from about 1% to about 20% by weight of the formulation; and (c) at least one viscosity regulating agent present in an amount of from about 0.5% to about 10% by weight of the formulation. In any embodiments, the pregnenolone may be loaded onto a surface of a porous excipient located inside pores of the porous excipient. In any embodiments, the subject may be a human, a non-human primate, a dog, a cat, a cow, a sheep, a horse, or a rabbit. In any embodiments, the subject may be suffering from a disease or condition associated with decreased acetylcholine activity in the brain. In any embodiments, the disease or condition may be selected from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders. In any embodiments, the use may be effective to improve cognitive function such as memory and learning deficits.

Also provided are uses of pregnenolone in the preparation of a medicament for ipsilaterally increasing acetylcholine activity in brain tissue of a subject in need thereof, particularly a non-rodent subject, or for treating a disease or condition in a subject in need thereof selected from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders in need thereof, wherein the medicament is a pharmaceutical composition adapted for intranasal administration comprising an effective amount of pregnenolone in a pharmaceutically acceptable carrier. In some embodiments, the medicament is adapted for intranasal administration to only one nostril of the subject.

### BRIEF DESCRIPTION OF THE DRAWINGS

FIG. 1 shows that administering pregnenolone into one nostril increases acetylcholine in the amygdala ipsilateral to this nostril, but not in the contralateral amygdala. The effects of lateralized intranasal administration of pregnenolone on extracellular acetylcholine levels in the amygdala were measured by in vivo microdialysis in anesthetized rats. Values are presented as % of baseline with six baseline samples taken as 100 (mean+SE). Pregnenolone was administered at a concentration of 11.2 mg/mL in an oil-based formulation (vehicle). 5 μl of drug formulation was administrated intranasally in one nostril (ipsilateral hemisphere) and 5 μl vehicle was administered in the other nostril (contralateral hemisphere). The intranasal administration was performed at the 0 minute, time point. The graph shows the level of acetylcholine released in the amygdala in the two hemispheres before and after administration. There are statistically significant differences (p<0.005) between the ipsilateral hemisphere group and contralateral group at different time points (10, 20, 30, 40, 50, 60, 70, 80, 90, 100 minutes after drug treatment).

FIG. 2 shows that ipsilateral acetylcholine release can be achieved either by administering pregnenolone in only the left or in only the right nostril. The effects of lateralized intranasal administration of pregnenolone on extracellular acetylcholine levels in the ipsilateral amygdala were measured by in vivo microdialysis in anesthetized rats. Values are presented as % of baseline with six baseline samples taken as 100 (mean+SE). Pregnenolone was administered at a concentration of 11.2 mg/mL in a lipid-based formulation (vehicle). 5 μl of drug formulation was administrated intranasally in one nostril (ipsilateral hemisphere) and 5 μl vehicle was administered in the other nostril (contralateral hemisphere). The intranasal administration was performed at the 0 minute, time point. The graph shows the level of acetylcholine released in the amygdala in the ipsilateral (right and left) hemispheres before and after administration. There are no statistical significances (p>0.05) between the right ipsilateral hemisphere group and left ipsilateral group after the drug treatment. n=7 for the right ipsilateral amygdala; n=3 for the left ipsilateral amygdala.

FIG. 3 shows the effects of intranasal administration of pregnenolone on extracellular acetylcholine levels in the frontal cortex (A), hippocampus (B) and amygdala (C), as measured by in vivo microdialysis in anesthetized animals. Acetylcholine concentration values are presented as % of baseline with six baseline samples taken as 100 (mean+SE). Pregnenolone was administrated intranasally at a volume of 5 μl each in both nostrils, at the 0 minute time point. Time is presented in the x-axis and mean and standard error of acetylcholine concentration (expressed as % of baseline) are presented in the y-axis. Filled black circles represent the vehicle, unfilled white circles represent the 5.6 mg/mL pregnenolone dose and the triangle represents the 11.2 mg/mL pregnenolone dose.

FIG. 4 shows a schematic illustration of the microdialysis probe design. The semipermeable membrane allows molecules smaller than 6 KDa to pass through. The length of the active membrane is 2 mm for the frontal cortex and amygdala, and 4 mm for the hippocampus.

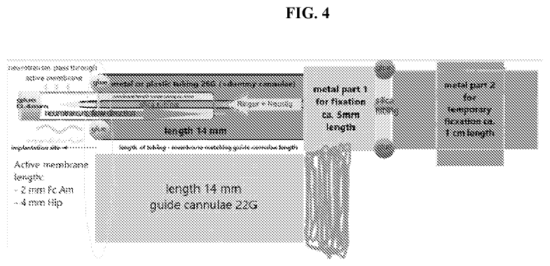

### DETAILED DESCRIPTION

Described herein are compositions and methods for ipsilaterally increasing acetylcholine activity in brain tissue of a subject in need thereof. The methods comprise administering pregnenolone intranasally. In some embodiments, the pregnenolone is administered only to one nostril, and acetylcholine activity is increased in the brain hemisphere ipsilateral to said nostril. In some embodiments, the methods are for treating diseases or disorders associated with acetylcholine deficiency, such as schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders. In some embodiments, the method is for improving cognitive function such as memory and learning deficits.

### I. Definitions

Technical and scientific terms used herein have the meanings commonly understood by one of ordinary skill in the art to which the present invention pertains, unless otherwise defined. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, “subject” denotes any non-rodent mammal, including humans. The subject may be in need increased acetylcholine activity in the brain, including being in need of increased acetylcholine activity in only one hemisphere of the brain. The subject may be in need of treatment for a disease or disorder associated with reduced acetylcholine activity in the brain, including a disease or disorder associated with reduced acetylcholine activity in only one hemisphere of the brain. For example, a subject may be suffering from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders.

As used herein, “ipsilateral” or “ipsilaterally” is a relative term used to specify the region of the brain located at the same side as a particular nostril of a subject. For example, the right side of the brain is ipsilateral to the right nostril.

As used herein, “contralateral” or “contralaterally” is a relative term used to specify the region of the brain located at the opposite side of one the nostril of a subject. For example, the right side of the brain is contralateral to the left nostril.

As used herein, the term “administering” includes directly administering to another, self-administering, and prescribing or directing the administration of an agent as disclosed herein.

As used herein, the phrases “effective amount” and “therapeutically effective amount” mean that active agent dosage or plasma concentration in a subject, respectively, that provides the specific pharmacological effect for which the active agent is administered in a subject in need of such treatment. It is emphasized that an effective amount of an active agent will not always be effective in treating the conditions/diseases described herein, even though such dosage is deemed to be an effective amount by those of skill in the art.

As used herein, the term “pharmaceutical composition” refers to one or more active agents formulated with a pharmaceutically acceptable carrier, excipient or diluent.

The phrase “pharmaceutically acceptable” is employed herein to refer to those compounds, materials, compositions, and/or dosage forms which are, within the scope of sound medical judgment, suitable for use in vivo without excessive toxicity, irritation, allergic response, or other problem or complication, commensurate with a reasonable benefit/risk ratio.

The citation or identification of any document herein is not an admission that such document is prior art to the present invention.

### II. Pregnenolone

Pregnenolone (PREG) can be used to mimic the function of endogenous neurosteroids to induce acetylcholine release. PREG is synthesized both in the central nervous system and in the peripheral nervous system from cholesterol by the cytochrome P450 cholesterol side-chain cleavage enzyme (CYP450scc), which is expressed in astrocytes and neurons. PREG can be converted into different neuroactive steroids such as DHEA, testosterone, progesterone, estrogen and cortisol. See Melcangi, R. C. et al., “Role of neuroactive steroids in the peripheral nervous system,” Front. Endocrinol., 2, 104 (2011). PREG may also naturally be converted to pregnenolone sulfate (PREG-S) by a sulfotransferase. See Robel, P. et al., “Biosynthesis and assay of neurosteroids in rats and mice: functional correlates,” J. Steroid Biochem. Mol. Biol., 53, 355-360 (1995); Dufort, I. et al., “Isolation and characterization of a stereospecific 3beta-hydroxysteriod sulfotransferase (pregnenolone sulfotransferase) cDNA,” DNA Cell Biol., 15, 481-487 (1996); Kohjitani, A. et al., “Regulation of SULT2B1a (pregnenolone sulfotransferase) expression in rat C6 glioma cells: relevance of AMPA receptor-mediated NO signaling,” Neurosci. Lett., 430, 75-80 (2008).

PREG-S both inhibits (through negative modulation of γ-aminobutyric acid (GABA_{A}) receptors) and activates (through positive modulation of N-methyl-D-aspartate (NMDA) receptors) the medial septum diagonal band cholinergic neurons, which project to the hippocampus. See Flood, J. F. et al., “Pregnenolone sulfate enhances post-training memory processes when injected in very low doses into limbic system structures: the amygdala is by far the most sensitive,” Proc. Natl. Acad. Sci. U.S.A., 92, 10806-10810 (1995). It has been reported that PREG-S can increase acetylcholine activity, a central neurotransmitter in the cholinergic transmission involved in memory processes, and PREG-S administration has been reported to enhance memory in aging rats. See Vallde, M. et al., “Steroid structure and pharmacological properties determine the anti-amnesic effects of pregnenolone sulphate in the passive avoidance task in rats,” Eur. J Neurosci., 14, 2003-2010 (2001).

It has also been reported that PREG by itself or by its natural conversion into PREG-S can improve memory. See Liyou, N. E. et al., “Localization of a brain sulfotransferase, SULT4A1, in the human and rat brain: an immunohistochemical study,” J. Histochem. Cytochem. Off J. Histochem. Soc., 51, 1655-1664 (2003); Salman, E. D. et al., “Expression and localization of cytosolic sulfotransferase (SULT) 1A1 and SULT1A3 in normal human brain,” Drug Metab. Dispos. Biol. Fate Chem., 37, 706-709 (2009); Nuwayhid and Werling, “Steroids modulate N-methyl-D-aspartate-stimulated [3H] dopamine release from rat striatum via sigma receptors,” J. Pharmacol. Exp. Ther., 306, 934-940 (2003). For example, Nuwayid and Werling (2003, supra) reported that PREG inhibited NMDA-stimulated [³H] dopamine release in the striatum via sigma receptors and that the coupled-PKCβ pathway is also involved.

The medical use of PREG and PREG-S for treating CNS disorders requires development of controlled and targeted delivery systems of these drugs to specific brain tissue. Prior attempts of delivering PREG or PREG-S to brain tissue involved systemic delivery of the drugs via intracerebroventricular injection. For example, Flood, J. F. et al., “Memory-enhancing effects in male mice of pregnenolone and steroids metabolically derived from it,” Proc. Natl. Acad. Sci. U.S.A., 89, 1567-1571 (1992) reported a memory-enhancing effect in mice by immediate post-training intracerebroventricular administration of PREG-S. Intracerebroventricular injection of PREG-S in rodents was reported to compensate for scopolamine-induced learning deficits in visual discrimination in Meziane, H. et al., “The neurosteroid pregnenolone sulfate reduces learning deficits induced by scopolamine and has promnestic effects in mice,” Psychopharmacology (Berl.), 126, 323-330 (1996). In addition, intraperitoneal or bilateral intrahippocampal injection of PREG-S was reported to transiently corrected memory deficit in rats in Vallde, M. et al., “Neurosteroids: deficient cognitive performance in aged rats depends on low pregnenolone sulfate levels in the hippocampus,” Proc. Natl. Acad. Sci. U.S.A., 94, 14865-14870 (1997). Intracerebroventricular injection of PREG-S was reported to improve spatial memory concomitantly with an increase in acetylcholine release in the hippocampus in Darnauddry, M., et al., “Pregnenolone sulfate increases hippocampal acetylcholine release and spatial recognition,” Brain Res., 852, 173-179 (2000).

It also has been reported that oral administration of pregnenolone to human patients suffering from schizoprehnia improved cognitive function. See Marx C. E. et al., “Proof-of-concept trial with the neurosteroid pregnenolone targeting cognitive and negative symptoms in schizophrenia,” Neurophsycopharmacology, 34: 1885-903 (2009).

Intranasal delivery of pregnenolone was reported in Ducharme, N. et al., “Brain distribution and behavioral effects of progesterone and pregnenolone after intranasal or intravenous administration,” Eur. J. Pharmacol., 641, 128-134 (2010). In Ducharme, N. et al. (2010), radioactively labeled pregnenolone was administered intranasally to both nostrils of the mice and delivery of the pregnenolone to blood and brain was examined by measuring radioactively labeled pregnenolone. This study showed that intranasal administration of pregnenolone to both nostrils of mice resulted in uptake of pregnenolone into blood, and varying levels of pregnenolone were observed in all examined areas of the brain. Thus, according to Ducharme, N. et al. (2010), administration of pregnenolone to both nostrils resulted in systemic delivery of pregnenolone to blood and both hemispheres of the brain.

### III. Intranasal Administration of Pregnenolone for Ipsilaterally Increasing Acetylcholine Activity

As noted above, described herein are compositions and methods for ipsilaterally increasing acetylcholine activity in brain tissue of a subject in need thereof. The ability to selectively increase acetylcholine activity in one hemisphere of the brain has not been heretofore described, and offers distinct advantages where increased acetylcholine activity is desired in a specific area of the brain, such as in a particular hemisphere of the brain, such as may arise in the context of stroke, Schizophrenia, depression, Parkinson's disease and Alzheimers' disease.

The methods described herein are based on the surprising discovery that intranasal delivery of pregnenolone into only one nostril of the subject increases acetylcholine activity only in the hemisphere of the brain ipsilateral to that nostril. Although it has previously been reported that pregnenolone can be delivered to the brain intranasally to improve cognitive functions in rodent animal models, these studies suggested that pregnenolone was delivered systemically through the blood brain barrier. See Ducharme, N. et al., “Brain distribution and behavioral effects of progesterone and pregnenolone after intranasal or intravenous administration,” Eur. J. Pharmacol., 641, 128-134 (2010); Abdel-Hafiz, L., et al., “Promnestic effects of intranasally applied pregnenolone in rats,” Neurobiol. Learn. Mem., 133, 185-195 (2016). Indeed, the pregnenolone distribution observed in Ducharme et al. (2010, supra) is consistent with pregnenolone being a small lipophilic drug that would easily cross the blood-brain barrier. It was therefore highly unexpected that administering pregnenolone in only one nostril could increase acetylcholine activity in only the ipsilateral hemisphere, because a drug crossing the blood-brain barrier would not be expected to selectively target one hemisphere over the other.

Accordingly, some aspects of the invention relate to methods that comprise administering pregnenolone only to one nostril, and achieve an increase in acetylcholine activity in the ipsilateral brain hemisphere of said nostril. In some embodiments, acetylcholine activity is not substantially increased in a contralateral brain hemisphere of said nostril. As used herein, “not substantially increased” means that the measured parameter is not statistically significantly increased after administration of pregnenolone compared to before administration of pregnenolone. Thus, there is no substantial increase in acetylcholine activity if the acetylcholine activity is not statistically significantly increased after administration of pregnenolone compared to before administration.

The methods are effective to increase acetylcholine activity in either hemisphere. That is, acetylcholine activity can be increased in the left hemisphere by administering pregnenolone in the left nostril, and acetylcholine activity can be increased in the right hemisphere by administering pregnenolone in the right nostril.

In some embodiments, the method results in increased acetylcholine activity in the hippocampus.

In some embodiments, the method results in increased acetylcholine activity in ipsilateral amygdala. In some embodiments, administering pregnenolone in a nostril increases acetylcholine activity in the ipsilateral amygdala within 1 minute, 5 minutes, 10 minutes, 20 minutes, 30 minutes, 40 minutes, 50 minutes, or 60 minutes of the administration. In some embodiments, administering pregnenolone in a nostril increases acetylcholine activity in the ipsilateral amygdala within 10 minutes of the administration. In some embodiments, acetylcholine activity in the ipsilateral amygdala remains increased as compared to initial levels for at least 10 minutes, 20 minutes, 30 minutes, 40 minutes, 50 minutes, 60 minutes, 70 minutes, 80 minutes, 90 minutes, 100 minutes, 110 minutes, 120 minutes, 130 minutes, 140 minutes, 150 minutes, 160 minutes, 170 minutes, 180 minutes, 190 minutes, 200 minutes, or 210 minutes. In some embodiments, acetylcholine activity in the ipsilateral amygdala remains increased as compared to initial levels for at least 100 minutes.

Without being bound by theory, the rapid effect of intranasal pregnenolone administration suggests that pregnenolone is transported via a direct olfactory/trigeminal nerve pathway rather than (or in addition to) crossing the blood-brain barrier, because crossing the blood-brain barrier is expected to require more time. See, e.g. Wang, Y. et al., “Brain uptake of dihydroergotamine after intravenous and nasal administration in the rat,” Biopharm. Drug Dispos., 19, 571-575 (1998); Chou, K.-J. and Donovan, M. D., “Lidocaine distribution into the CNS following nasal and arterial delivery: a comparison of local sampling and microdialysis techniques,” Int. J. Pharm., 171, 53-61(1998); Sakane, T. et al., “Transport of cephalexin to the cerebrospinal fluid directly from the nasal cavity,” J. Pharm. Pharmacol., 43, 449-451 (1991). Without being bound by theory, the ipsilateral specificity of the methods described herein also is consistent with transport via olfactory or trigeminal neuronal pathways.

### IV. Compositions for Intranasal Administration of Pregnenolone

In accordance with the methods described herein, pregnenolone can be administered intranasally in any composition suitable for intranasal administration, such as a composition comprising pregnenolone and a pharmaceutically acceptable carrier for intranasal administration.

The pregnenolone can be pregnenolone per se, which is hydrophobic, or the sulfated derivative, pregnenolone sulfate, which is water-soluble, can be used.

Compositions suitable for intranasal administration include solutions, suspensions, or powder formulations of pregnenolone in a pharmaceutically acceptable carrier suitable for intranasal administration. A composition for intranasal administration may be an aqueous formulation, including an aqueous solution, aqueous gel, aqueous suspension, aqueous liposomal dispersion, aqueous emulsion, aqueous microemulsion, and combinations thereof. Alternatively, an intranasal composition may be a non-aqueous formulation, such a non-aqueous solution, non-aqueous gel, non-aqueous suspension, non-aqueous liposomal dispersion, non-aqueous emulsion, non-aqueous microemulsion, and combinations thereof. The intranasal composition may include an aqueous component and a non-aqueous component. Alternatively, a composition suitable for intranasal administration may be a powder formulation. A powder formulation may be a simple powder mixture, powder microsphere, coated powder microsphere, liposomal dispersions, and combinations thereof.

In accordance with any embodiments, the intranasal composition may also include an excipient having bio-adhesive properties.

The formulation may include one or more organic solvents, suspending agents, isotonicity agents, buffers, emulsifiers, stabilizers, and preservatives.

In some embodiments, the pregnenolone is formulated in an oleogel intranasal pharmaceutical compositions, such as described in U.S. Pat. No. 8,574,622 for testosterone, such as a composition that includes the active agent(s) and that further comprises (a) at least one lipophilic or partly lipophilic carrier present in an amount of from about 60% to about 98% by weight of the formulation; (b) at least one compound having surface tension decreasing activity present in an amount of from about 1% to about 20% by weight of the formulation; and (c) at least one viscosity regulating agent present in an amount of from about 0.5% to about 10% by weight of the formulation.

In such oleogel embodiments, the lipophilic or partly lipophilic carrier may be any such carrier suitable as a carrier or vehicle for a nasal pharmaceutical composition, such as an oil, such as a vegetable oil, such as castor oil, hydrogenated castor oil, soybean oil, sesame oil, or peanut oil, or any vehicle discussed below that is lipophilic or partly lipophilic, or any other suitable lipophilic or partly lipophilic carrier.

In such oleogel embodiments, the compound(s) having surface tension decreasing activity may be one or more surfactants such as lecithin, fatty acid esters of polyvalent alcohols, of sorbitanes, of polyoxyethylensorbitans, of polyoxyethylene, of sucrose, of polyglycerol and/or one or more humectants such as sorbitol, glycerine, polyethylene glycol, and macrogol glycerol fatty acid esters, or one or more oleoyl macrogolglycerides (such as LABRAFIL® M 1944 CS, available from Gattefosse (France), or any surfactant discussed below, or any other suitable surfactant.

In such oleogel embodiments, the viscosity regulating agent(s) may be one or more selected from thickeners and gelling agents, such as cellulose and cellulose derivatives, polysaccharides, carbomers, polyvinyl alcohols, povidone, colloidal silicon dioxide, cetyl alcohols, stearic acid, beeswax, petrolatum, triglycerides and lanolin, or any viscosity regulating agent discussed below, or any other suitable surfactant.

Additionally or alternatively, the pregnenolone may be formulated in an intranasal pharmaceutical composition as described in U.S. Patent Application Publication US 2018/0008615, such as an intranasal pharmaceutical compositions wherein the pregnenolone is loaded onto a porous agent. In such embodiments, the pregnenolone may be loaded onto a surface of a porous agent located inside pores of the porous agent. As described in US 2018/0008615, the active-agent loaded porous agent may itself be formulated in an oleogel composition, such as described those in U.S. Pat. No. 8,574,622.

In such porous agent embodiments, the porous agent may comprise an inorganic porous material, such as colloidal silicon dioxide, micro-porous silicon dioxide, meso-porous silicon dioxide, macro-porous silicon dioxide, polyorganosiloxanes, pharmaceutical clays, silicon dioxide nanotubes, silicon dioxide gel, magnesium alumosilicate (such as but not limited to VEEGUM® from Vanderbilt Minerals, LLC), activated carbon, anhydrous calcium phosphate, calcium carbonate, alumina, and combinations of any two or more thereof. Exemplary inorganic porous materials include porous silicon dioxide commercially available under the SYLOID® brand from W.R. Grace & Co. (such as but not limited to SYLOID® 244FP, 72FP, XDP6035 (also known as SILSOL™ 6035), XDP3050, XDP3150, AL-1FP, and combinations of any two or more thereof), porous silicon dioxide available under the AEROPERL® brand from Evonik Industries, Corp. (such as but not limited to AEROPERL® 300, which has a surface area of about 260 to 320 m²/g (such as about 300 m²/g), a pore volume of about 1.5 to 1.9 ml/g, and an average particle size of about 20 to about 60 μm), silicon dioxide PARTECK® SLC from EMD Millipore, NEUSILIN® (a synthetic, amorphous form of magnesium aluminometasilicate) from Fuji Chemical Industry, Zeolite Socony Mobil-5, Mobil Composition of Matter No. 41, SBA-15, FDU-11, OMS-7, OMS-Lemon-7, and IITM-56. In some embodiments, the porous agent comprises silicon-based powders, which may be hydrophobic or hydrophilic, e.g., depending on groups chemically bonded to their surfaces.

In some embodiments, the porous agent comprises an organic-inorganic hybrid, such as metal-organic frameworks (MOFs). Exemplary hybrid materials can be formed by self-assembly of polydentate bridging ligands and metal connecting points.

In some embodiments, the porous agent comprises organic polymers, such as microporous organic polymers, polystyrene, cellulose, and/or poly(methyl methacrylate). In some embodiments, microporous organic polymers are formed by carbon-carbon coupling reactions and comprised of non-metallic elements such as carbon, hydrogen, oxygen, nitrogen, and/or boron. In some embodiments, organic polymers are produced by emulsion polymerization and hypercrosslinking followed by chemical etching of sacrificial SiO₂ cores. In some embodiments, networks of organic polymers are constructed from small organic building blocks.

In some embodiments, the porous agent comprises porous materials based on complexing agents, such as an ion exchange resin (such as but not limited to cross-linked polystyrene) or an adsorbent (such as but not limited to β-cyclodextrin-based porous silica, α-cyclodextrin-based porous silica, hydroxpropyl-β-cyclodextrin-based porous silica, and porous materials based on other adsorbent resins).

In some embodiments, the surface of the porous agent-including the inner pore surface—is functionalized to bind the active agent(s) and/or control release of the active agent(s) after a certain amount of time or in response to a stimulus.

The active agent-loaded porous agent may be formulated in any vehicle suitable as a vehicle for a nasal pharmaceutical composition. In some embodiments, the vehicle for the porous agent is a hydrophilic vehicle. In some embodiments, the vehicle is a lipophilic or partly lipophilic vehicle, such as a vehicle comprising one or more fats, oils, waxes, phospholipids, steroids (e.g., cholesterol), sphingolipids, ceramides, sphingosines, prostaglandins, and/or fat-oil vitamins. In some embodiments, the vehicle comprises an oil or a mixture of oils, such as vegetable oil, castor oil, hydrogenated castor oil, soybean oil, sesame oil, or peanut oil; fatty acid esters, such as ethyl- and oleyl-oleate, isopropylmyristate; medium chain triglycerides; glycerol esters of fatty acids; polyethylene glycol; phospholipids; white soft paraffin; or combinations of any two or more thereof.

The vehicle may be present in any suitable amount, such as an amount effective to provide desired properties for nasal administration, desired physical properties, desired release properties, desired pharmacokinetics, etc. In some embodiments, the composition comprises a vehicle in an amount of from about 15% to about 98% by weight, about 30 to about 98% by weight, about 50% to about 95% by weight, about 75% to about 95% by weight, about 80%, or about 90% by weight, based on the total weight of the composition. In some embodiments, the composition comprises a vehicle in an amount of from 15% to 98% by weight, 30 to 98% by weight, 50% to 95% by weight, 75% to 95% by weight, 80%, or 90% by weight, based on the total weight of the composition.

The active agent-loaded porous agent may be formulated with one or more compounds having surface decreasing activity, e.g., surfactants. The surfactant, if present, may be any surfactant suitable for use as a surfactant in a nasal pharmaceutical composition. In some embodiments, the surfactant is selected from anionic, cationic, amphoteric, and non-ionic surfactants, including, but not limited to, lecithin, fatty acid esters of polyvalent alcohols, fatty acid esters of sorbitanes, fatty acid esters of polyoxyethylensorbitans, fatty acid esters of polyoxyethylene, fatty acid esters of sucrose, fatty acid esters of polyglycerol, oleoyl polyoxylglycerides (such as but not limited to apricot kernel oil PEG-6-esters), oleoyl macrogolglycerides, and/or humectants such as sorbitol, glycerine, polyethylene glycol, macrogol glycerol fatty acid ester, and combinations of any two or more thereof. In some embodiments, the surfactant comprises an oleoyl macrogolglyceride (such as LABRAFIL® M 1944 CS (Gattefosse, Saint-Priest, France)) or a mixture of oleoyl macrogolglycerides.

The active agent-loaded porous agent may be formulated with one or more viscosity-regulating agents, which may be any viscosity-regulating agent suitable for use as a viscosity-regulating agent in a nasal pharmaceutical composition. In some embodiments, the viscosity-regulating agent comprises mesoporous silica (which may be loaded with active agent or unloaded). In some embodiments, the viscosity-regulating agent comprises cellulose, cellulose-containing substances, polysaccharides, carbomers, polyvinyl alcohol, povidone, colloidal silicon dioxide, cetyl alcohols, stearic acid, beeswax, petrolatum, triglycerides, lanolin, or combinations of any two or more thereof. In some embodiments, the viscosity-regulating agent comprises colloidal silicon dioxide (such as but not limited to AEROSIL® 200 (Evonik) and/or CAB-O-SIL® M5 (Cabot)). In some embodiments, the viscosity-regulating agent comprises synthetic silica, such as SYLODENT® (precipitated silica with a compacted bulk density of about 110 kg/m³, a specific surface area of about 190 m²/g, and an average particle size of about 18 μm) or SYLOBLANC® silicas (porous silica gel with a pore volume of about 1.6 ml/g and an average particle size of about 3 μm) from W.R. Grace & Co. In some embodiments, the viscosity-regulating agent comprises hydrophilic fumed silica, such as AEROSIL® 200 and/or lipophilic silicon dioxide, such as AEROSIL® R972 (which is fumed silica after-treated with dimethyldichlorosilane, and which has a surface area of about 90 to about 130 m²/g). Without being bound by theory, it is believed that hydrophilic fumed silica can be used to prepare a thixotropic gel composition with a high temperature stability as compared to a comparable gel produced with other viscosity-regulating agents.

The viscosity-regulating agent, if present, may be present in an amount effective to adjust the viscosity of the composition to the desired level. In some embodiments, the composition comprises from about 0.5 to about 20% by weight, about 0.5 to about 10% by weight, about 0.5 to about 7% by weight, about 1 to about 4% by weight, about 4% by weight, or about 2% by weight viscosity-regulating agent, based on the total weight of the composition. In some embodiments, the composition comprises from 0.5 to 20% by weight, 0.5 to 10% by weight, 0.5 to 7% by weight, 1 to 4% by weight, 4% by weight, or 2% by weight viscosity-regulating agent, based on the total weight of the composition.

Regardless of the specific formulation used, the pregnenolone is formulated to provide a therapeutically effective amount of the active agents in doses suitable for the route of administration, such as a volume of composition suitable for administration to one or both nostrils.

### V. Therapeutic Methods and Uses

Described herein are therapeutic methods for ipsilaterally increasing acetylcholine activity in brain tissue of a subject in need thereof, such as for increasing acetylcholine activity only in one hemisphere in the brain, as well as pregnenolone formulations for use in such methods.

In some embodiments, the methods comprise administering pregnenolone intranasally to a subject in need thereof. In some embodiments, the pregnenolone is administered only to one nostril of the subject.

In some embodiments, the subject is suffering from a disease or disorder associated with acetylcholine deficiency, such as schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders. In some embodiments, the subject is in need of improvement of cognitive function, such as in need of treatment for memory and/or learning deficits.

As noted above, the subject may be any non-rodent mammal, such as a human, non-human primate, dog, cat, cow, sheep, horse, or rabbit.

As also noted above, the pregnenolone can be administered in any pharmaceutical composition suitable for or adapted for intranasal administration.

As also noted above, the pregnenolone can be administered in an amount effective to increase acetylcholine activity, as discussed above. As used herein, the term “acetylcholine activity” refers to the release of acetylcholine in brain tissue. The release of acetylcholine in brain tissue can be assessed by methods such microdialysis and acetylcholine assays as described in the examples below, although the methods described herein are not limited by these or other specific methodologies for assessing acetylcholine activity.

In some embodiments, the pregnenolone is administered at a dose of from about 0.01 to about 2.0 mg per kilogram of bodyweight of the subject. That is, in some embodiments, a dose of from about 0.01 to about 2.0 mg per kilogram of bodyweight of the subject is effective to increase acetylcholine activity.

As noted above, in some embodiments, the method is effective to increase acetylcholine activity within 1 minute, 5 minutes, 10 minutes, 20 minutes, 30 minutes, 40 minutes, 50 minutes, or 60 minutes of the administration. In some embodiments, administering pregnenolone in a nostril increases acetylcholine activity in the ipsilateral amygdala within 10 minutes of the administration. In some embodiments, acetylcholine activity in the ipsilateral amygdala remains increased as compared to initial levels for at least 10 minutes, 20 minutes, 30 minutes, 40 minutes, 50 minutes, 60 minutes, 70 minutes, 80 minutes, 90 minutes, 100 minutes, 110 minutes, 120 minutes, 130 minutes, 140 minutes, 150 minutes, 160 minutes, 170 minutes, 180 minutes, for at least 190 minutes, 200 minutes, or 210 minutes. In some embodiments, the amount of pregnenolone is effective to sustain increased acetylcholine activity in brain tissue for at least 100 minutes.

The following examples are provided to illustrate the invention, but it should be understood that the invention is not limited to the specific conditions or details of these examples.

### VI. Uses

Also provided are pregnenolone formulations for use in ipsilaterally increasing acetylcholine activity in brain tissue of a subject in need thereof, or for use in treating a disease or condition selected from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders in a subject in need thereof. In some embodiments, the subject is a non-rodent subject. The pregnenolone formulation may be any pregnenolone formulation, including any pregnenolone formulation as described herein, that is suitable for use as pharmaceutical compositions and adapted for intranasal administration, comprising an effective amount of pregnenolone in a pharmaceutically acceptable carrier. In some embodiments, the pregnenolone formulation is adapted for intranasal administration to only one nostril of the subject.

In some embodiments, the pregnenolone formulation is administered only to one nostril, and acetylcholine activity is increased in an ipsilateral brain hemisphere of said nostril. In some embodiments, acetylcholine activity is not substantially increased in a contralateral brain hemisphere of said nostril. In some embodiments, the use additionally or alternatively results in increased acetylcholine activity in amygdala of the subject. In some embodiments, the use additionally or alternatively results in increased acetylcholine activity in hippocampus of the subject. In some embodiments, the acetylcholine activity is increased within 10 minutes. In some embodiments, acetylcholine activity in the brain tissue is sustained for at least 60 minutes. In some embodiments, acetylcholine activity in the brain tissue is sustained for at least 100 minutes.

In some embodiments, the effective amount of pregnenolone is from about 0.01 mg to about 2.0 mg per kilogram of bodyweight of the subject. In any embodiments, the pharmaceutically acceptable carrier may comprise (a) at least one lipophilic or partly lipophilic carrier present in an amount of from about 60% to about 98% by weight of the formulation; (b) at least one compound having surface tension decreasing activity present in an amount of from about 1% to about 20% by weight of the formulation; and (c) at least one viscosity regulating agent present in an amount of from about 0.5% to about 10% by weight of the formulation. In any embodiments, the pregnenolone may be loaded onto a surface of a porous excipient located inside pores of the porous excipient.

In any embodiments, the subject may be a human, a non-human primate, a dog, a cat, a cow, a sheep, a horse, or a rabbit. In any embodiments, the subject may be suffering from a disease or condition associated with decreased acetylcholine activity in the brain. In any embodiments, the disease or condition may be selected from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders. In any embodiments, the use may be effective to improve cognitive function such as memory and learning deficits.

Also provided are uses of pregnenolone in the preparation of a medicament for ipsilaterally increasing acetylcholine activity in brain tissue of a subject in need thereof, or for treating a disease or condition selected from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders in need thereof. In some embodiments, the subject is a non-rodent subject. The medicament may be any pregnenolone formulation, including any pregnenolone formulation as described herein, that is suitable for use as pharmaceutical compositions and adapted for intranasal administration, comprising an effective amount of pregnenolone in a pharmaceutically acceptable carrier. In some embodiments, the medicament is adapted for intranasal administration to only one nostril of the subject.

### Examples

### Materials and Methods

Subjects. A total number of 10 adult male Wistar rats 3-4 months of age and weighting between 400 and 500 grams, at the time of surgery, were obtained from the local animal facility (Tierversuchsanlage, University of Dusseldorf, Germany). They were grouped 4 per cage (Makrolon cage, type IV, 60.0×20.0×38.0 centimeter), and separated into individual cases after the surgery. They were housed under a reversed light-dark cycle (light off from 7 after midnight (AM) to 7 post meridiem (PM)), with free access to food and water. The room temperature is 20±2 Celsius degrees and the environment had a controlled humidity. After two weeks of adaptation, animals underwent microdialysis as described below. All experiments were carried out in accordance with the European Communities Council Directive (86/609/EEC) on animal welfare, and approved by the German Animal Protection Law Authorities—LANUV Nordrhein-Westfalen.

Surgery. Rats underwent implantation of microdialysis probes into specific brain areas. They were anesthetized with a mixture of ketamine hydrochloride (90.0 milligram/kilogram (mg/kg); Pharmacia & Upjohn) and xylazine hydrochloride (8.0 mg/kg; Bayer) and placed in a stereotaxic frame (David Kopf Instruments). Additionally, Bupivacaine (2.5 milligram/milliliter(mg/mL), injection volume 0.1 milliliter (mL) above the skull; Bucain, Deltaselect HmbH) was applied as a local anesthetic. Two guide cannulae (14 millimeter (mm) long, 26 gauge) for microdialysis probes were implanted in both the right and left amygdala (Anteriorposterior (AP):—2.5 mm; Mediolateral (ML): ±4.6 mm; Dorsoventral (DV):—7.2 mm). All coordinates were relative to bregma according to a rat brain atlas (Paxinos G, Watson C (1986) THE RAT BRAIN IN STEREOTACTIC COORDINATES (Academic, New York), 2nd Edition). For additional fixation of the implant, two stainless steel screws of 2.6 mm were fastened to the skull. In order to reduce postoperative pain, Carprofen (5 mg/kg Rimadyl, Pfizer) carried by phosphate-buffered saline (Dulbecco's Phophate Buffered Saline (PBS), Life Technologies Ltd) was injected into the head-neck area with an injection volume of 1 mg/kg (0.1 milliliter/kilogram carprofen and 0.9 milliliter/kilogram Phophate Buffered Saline (PBS)). The animals were allowed to recover from surgery for 3 to 6 days before the microdialysis was performed.

Microdialysis. Prior the microdialysis process, the animals were anesthetized with Urethane intraperitoneal injection. (1.25 gram/kilogram, Sigma Aldrich). To allow fluid supply (perfusion liquid) (Ringer's solution 0.2 milliliter every 20 minutes) without physical contact with the animal, a catheter was placed into the intraperitoneal cavity. The animal was placed in an acrylic box (45×25×22 centimeter), and the body temperature was monitored and held stable at 36.5±0.5 degrees Celsius by atemperature controller (CMA/150) and a heating pad. The inlet tubes were connected to a microinfusion pump (CMA/100) and were perfused with Ringer's solution containing Neostigmine (10 micromoles) with a flow rate of 2 microliter/minute (perfusate). The fluid rate of the fluid flowing through the probe was thus controlled by the syringe pump. Neostigmine is a cholinesterase inhibitor which was perfused in order to obtain levels of acetylcholine easily detectable with the HPLC method currently available (sensitivity limits 50-100 femtomole/injection). The inhibition of cholinesterase caused continuous occupation of muscarinic presynaptic inhibitory receptors, thereby maintaining an inhibitory tone which controlled acetylcholine release from the cholinergic terminal. de Boer, P. et al., “The effect of acetylcholinesterase inhibition on the release of acetylcholine from the striatum in vivo: interaction with autoreceptor responses,” Neurosci. Lett., 116, 357-360 (1990). The perfusate was designed to have a lower concentration compared to the area surrounding the probe, which ensured that the flow of the flux went into the probe and not the other way around. Once the fluid flowed through the membrane, the perfusion fluid, now dialysate, should reflect the concentration of the neurotransmitter of interest in the extracellular fluid in that area as described in Kho, C. M. et al., “A Review on Microdialysis Calibration Methods: the Theory and Current Related Efforts,” Mol. Neurobiol., 54, 3506-3527 (2017). After a 2 hour stabilization period, baseline samples, each corresponding to a time window of 10 minutes, were collected. After the sixth baseline sample, 5 microliter (μL) of 11.2 milligram/milliliter (mg/mL) pregnenolone (PREG) in one nostril (ipsilateral) and 5 μL of vehicle lipid gel in the opposite nostril (contralateral) were intranasally administered. After the treatment, another 10 samples, each also corresponding to a time window of 10 minutes, were collected. The volume collected for each sample was 20 μL. In order to determine changes in recovery (relation between concentration of acetylcholine in the probe surrounding area and that in the collected dialysate) caused by external factors that may decrease the efficiency of the probe, 10 μL of internal standard was present in each vial (25 μL of ethylhomocholine stock solution in 100 mL of NaOH diluent).

Microdialysis probes. The microdialysis probe was made of a fused silica open-ended tube attached with a semipermeable membrane as described in Boix, F. et al., “Substance P decreases extracellular concentrations of acetylcholine in neostriatum and nucleus accumbens in vivo: possible relevance for the central processing of reward and aversion,” Behav. Brain Res., 63, 213-219 (1994), and Boix, F. et al., “Relationship between dopamine release in nucleus accumbens and place preference induced by substance P injected into the nucleus basalis magnocellularis region. Neuroscience,” 64, 1045-1055 (1995). This membrane allowed molecules to go through its pores via diffusion. The size of the pores of the membrane was 6 kilodalton (kDa). A small segment of the membrane was placed inside of ⅓ of a metal tubing and they were glued together with glue 2 Tor Epoxy. The length of membrane outside of the silica (active membrane) was 2.4 mm. The tip of the membrane was then glued (0.4 mm) with glue 2 Tor Epoxy. Once the probe was produced, it was glued to the silica tube. Inside the probe, a fused silica capillary tube operated as an outlet. A metal socket was glued at a specific distance, to define a proper probe's length, according to the cannula's length.

Drugs. Pregnenolone (PREG) (Bayer HealthCare Pharmaceuticals) mixed in a lipid-based gel formulation was used. The composition of the gel formulation containing 11.2 mg/mL pregnenolone was 1.12% micronized pregnenolone, 90.88% castor oil, 4.0% oleoyl polyoxylglycerides, and 4.0% colloidal silicon dioxide. The composition of the 5.6 mg/mL pregnenolone gel formulation was 0.56% micronized pregnenolone, 91.44% castor oil, 4.0% oleoyl polyoxylglycerides, and 4.0% colloidal silicon dioxide. The gel formulations were made by adding micronized pregnenolone to castor oil, and mixing for 10 minutes at 13000 revolutions per minute (rpm). Then, oleoyl polyoxylglycerides were added and mixed for 2 minutes at 13000 rpm. Finally, colloidal silicon dioxide was added and mixed for 2 minutes at 13000 rpm. The same gel formulation without pregnenolone (gel vehicle) was used as a control. For every administration, each animal received 5 μL of the gel vehicle formulation in one nostril and 5 μL of the 11.2 mg/mL or of the 5.6 mg/mL pregnenolone (PREG), respectively, in the other nostril. Administration was performed with a Transferpettor pipette (GMBH+CO KG, Wertheim, Germany). Since the average weight of the animals at the surgery time was 450 gram, the dose used was 0.373 milligram/kilogram (mg/kg), meaning a total of 0.112 milligram/milliliter (mg/mL) per rat.

Acetylcholine assay. For the purpose of quantifying the amount of acetylcholine in the microdialysis samples, a high-pressure liquid chromatography (HPLC) technique with electrochemical detection (EC) was used as described in de Souza Silva, M. A. et al., “Differential modulation of frontal cortex acetylcholine by injection of substance P into the nucleus basalis magnocellularis region in the freely-moving vs. the anesthetized preparation,” Synap. N. Y. N, 38, 243-253 (2000). Acetylcholine was separated on a 75 mm long reverse-phase column filled with ChromSpher 5C18 (Merck KGaA, Darmstadt, Germany) and loaded with sodiumdodecylsulfate (Sigma-Aldrich, Saint Louis, Mo., US). Detection took place due to the use of an enzyme reactor coupled to the column. The enzyme reactor was filled with LiChrosorb-NH2 (Merck), activated by glutaraldehyde (Merck, Darmstadt, Germany), and then loaded with acetylcholineesterase (Sigma-Aldrich, Saint Louis, Mo., US). The enzymes were covalently bound to the stationary phase. The enzyme reactor converted acetylcholine to hydrogen peroxide, which was electrochemically detected at a platinum electrode set at a potential of 0.350 millivolt (mV). The reference electrode was an in situ Ag/AgCl (ISAAC) electrode (Antec, Fremont, Calif., US). The mobile phase was composed of 1 millimolar (mM) tetramethylammonium chloride and 0.18 molar (M) K2HPO4 and adjusted to pH 8.0 with KH2PO4 (Merck, Darmstadt, Germany) as described in de Souza Silva, M. A., et al., “Neurokinin3 receptor as a target to predict and improve learning and memory in the aged organism,” Proc. Natl. Acad. Sci. U.S.A, 110, 15097-15102 (2013).

The pH of the mobile phase (eluent), which flowed through the system, was controlled to pH=8 to facilitate the enzymatic conversions and to obtain better detection sensitivity. The mobile phase or eluent flowed at the rate of 0.3 microliter/minute (μl/min), using a high-pressure liquid chromatography (HPLC) pump (Merck, Darmstadt). The time required to complete a chromatogram was 8-9 minutes. The neurotransmitter content was analyzed with the help of Chrom Perfect Software (Justice Laboratory Software, Denville, N.J., USA).

Histology analysis. After the microdialysis process was finished, the rat was injected with a Phentobarbital overdose (0.5-1 milliliter (mL)) and perfusion with Phosphate Buffered Saline (PBS) and 10% formalin of the body was performed. The brain was carefully removed and placed in a vial with 10% formalin+30% sucrose (fixative) and stored at 4° C. for further histological analysis. Post-mortem histology was used to confirm the correct microdialysis implantation location. The brain was sliced with a cryostat (Leica CM1900) and located on gelatinized microscope glasses. The gelatinized microscope glasses, with Gelatine (Amresco), were previously prepared. Tissue Freezing Medium was used to fix the brain on the cryostat pedestal. All the brain was sliced, except for the cerebellum region. After one day, it was possible to perform the staining with cresyl violet (Sigma-Aldrich). The staining procedure required different dilutions of Ethanol (100%, 95%, 80%, or 70%), Cresyl violet dye solution and Xylol, as last step. The brain atlas (“The Rat Brain in Stereotaxic Coordinates—6th Edition,” 2017) was used to determine the accuracy of probe placement. Only the brains with successful cannulae implantation were considered in the statistical analysis.

Statistical analysis. The data from the high-pressure liquid chromatography (HPLC) analysis was further processed with IBM SPSS Statistics 24.0 software. Data from each brain area was analyzed. A two-way ANOVA was then performed for “side” within “time” factors. Moreover, graphs, depicting the concentration of acetylcholine in the amygdala hemispheres (ipsilateral and contralateral to the 11.2 mg/mL drug treatment) and changes over the time during the microdialysis process after the intranasal administrations, were then prepared using SigmaPlot 12.0.

### Example 1: Administering Pregnenolone Intranasally into One Nostril Ipsilaterally Increases Acetylcholine Activity

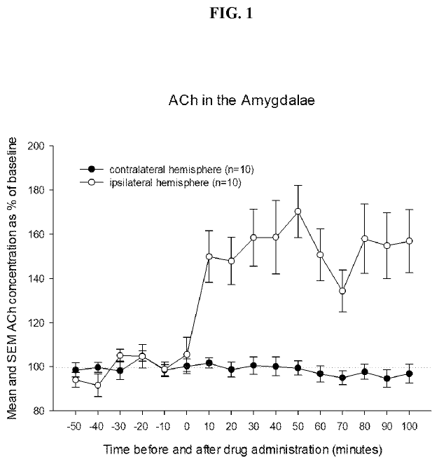

A two-way ANOVA pairwise comparison for repeated measures within subjects analysis of variance has been conducted to assess the impact of lateralized intranasal administration of pregnenolone (11.2 milligram/milliliter (mg/mL)), ipsilateral hemisphere, and vehicle, contralateral hemisphere, on release of extracellular acetylcholine (Ach) in the amygdala in each animal, as shown in FIG. 1. The number of animals investigated for analysis in the amygdalae is: n=10.

There was a statistically significant difference between extracellular release of Ach in the two amygdalae hemispheres (ipsilateral and contralateral) within each animal, F(1/9)=40.195; Wilks' Lambda=0.183, p<0.005, partial eta squared=0.817. Higher increase in the ipsilateral amygdala of extracellular level of acetylcholine was found.

Independent t-test (two-tailed) was performed to assess differences of extracellular acetylcholine (ACh) release in the two amygdala hemispheres, in the time points before intranasal administration and after intranasal administration, of 5 microliter (μl) 11.2 mg/mL pregnenolone (PREG) in one nostril (ipsilateral subgroup) and of 5 μl vehicle in the opposite nostril (contralateral group).

There were significant differences between the ipsilateral amygdala after 11.2 mg/mL administration and vehicle administration in the opposite nostril compared to the contralateral amygdala. These statistical significant differences were found at 10, 20, 30, 40, 50, 60, 70, 80, 90, and 100 minutes after treatment as indicated in table 1 below.

TABLE 1
Statistical Analysis of Results

| Time after | Difference in extracellular release | |
|---|---|---|
| administration | of acetylcholine (ipsilateral | |
| (min) | vs. contralateral amygdala) | |
| 10 | t(9.766) = | −4.024, p = 0.003 |
| 20 | t(18) = | −4.383, p = 0.0001 |
| 30 | t(10.662) = | −4.287, p = 0.001 |
| 40 | t(18) = | −3.411, p = 0.003. |
| 50 | t(10.295) = | −5.764, p = 0.0001 |
| 60 | t(10.722) = | −4.364, p = 0.001 |
| 70 | t(18) = | −3.911, p = 0.001 |
| 80 | t(18) = | −3.737, p = 0.002 |
| 90 | t(10.270) = | −3.896, p = 0.003 |
| 100 | t(18) = | −4.036, p = 0.001 |

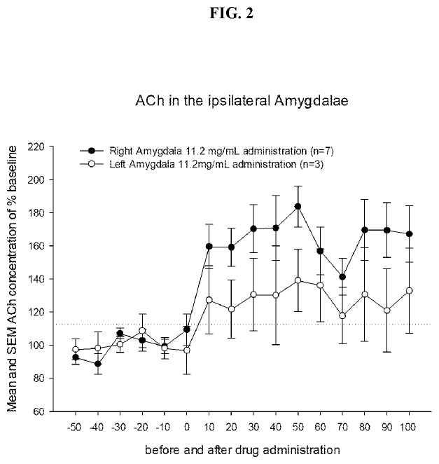

Furthermore, it was investigated whether there is a difference associated with intranasal delivery into the right versus left olfactory system. As shown in FIG. 2, when pregnenolone (PREG) was administered in the right nostril, acetylcholine (ACh) was released in the right amygdala, and when pregnenolone was administered in the left nostril, ACh was released in the left amygdala. No statistically significant difference was found between ACh releases upon pregnenolone administration into the left or right olfactory system, as depicted in FIG. 2. Thus, the ipsilateral increase in acetylcholine upon administration of pregnenolone in only one nostril is not limited to the right or left nostril, but rather, the pregnenolone can be administered in either nostril to achieve an ipsilateral increase in acetylcholine in the amygdala.

### Example 2: Administering Pregnenolone in Both Nostrils Increases Acetylcholine Activity in Both the Amygdala and in the Hippocampus

Both between-subjects and within-subjects analysis of variance were conducted to assess the impact of intranasal administration of pregnenolone (PREG) (5.6 milligram/milliliter (mg/mL), 11.2 milligram/milliliter (mg/mL)) or vehicle, in the frontal cortex, hippocampus and amygdala. The number of animals investigated for analysis of the frontal cortex were: n=7 for the vehicle group, n=5 for the PREG 5.6 mg/mL dose group, n=6 for the PREG 11.2 mg/mL dose group. The number of animals investigated for analysis for the hippocampus were: n=6 for the vehicle group, n=7 for the PREG 5.6 mg/mL dose group, n=5 for the PREG 11.2 mg/mL dose group. The number of animals investigated for analysis for the amygdala (animals 38 and 39 excluded) were: n=7 for the vehicle group, n=5 for the PREG 5.6 mg/mL dose group, n=4 for the PREG 11.2 mg/mL dose group.

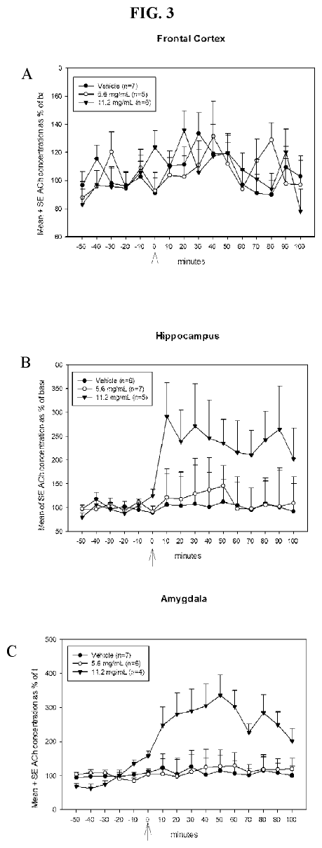

Neither pregnenolone (PREG) at dosage 5.6 mg/mL or 11.2 mg/mL had an effect on acetylcholine release in the frontal cortex, as shown in FIG. 3 A. There was no significant main effect for time, Wilks' Lambda=0.076, F(15,1)=0.810, p>0.05, partial eta squared=0.924, and no interaction between time and drug effect, Wilks' Lambda=0.021, F(30,2)=0.393, p>0.05, partial eta squared=0.924. Moreover, there was no significant main effect for drug F(2,15)=0.002, p>0.05, partial eta squared<0.001, as shown in FIG. 3A.

PREG at the 11.2 mg/mL dose had an effect on acetylcholine release in the hippocampus, but this effect was not significant. As in the amygdala, there is a second peak that may be due to additional transfer through systemic circulation/crossing the BBB. See FIG. 3B. PREG at the 5.6 mg/mL dose had no effect. There was no significant main effect for time, Wilks' Lambda=0.003, F(15,1)=19.415, p>0.05, partial eta squared=0.997. However, there was interaction between time and drug effect, Wilks' Lambda <0.001, F(30,2)=84.209, p<0.05, partial eta squared=0.999. Also, the difference between the high and low doses on acetylcholine release in the hippocampus was not significant F(15,2)=1.501 p>0.05, partial eta squared=0.167.

An interaction effect between time and drug was found, and one-way ANOVA was conducted to compare acetylcholine levels in the different time points after administrating the 11.2 mg/mL pregnenolone dose; Wilks' Lambda=0.01, F(4,1)=232.984, p<0.05. One-way ANOVA for the 5.6 mg/mL dose and vehicle drug showed no significant results; Wilks' Lambda=0.086, F(4,1)=1.762, p>0.05 and Wilks' Lambda=0.104, F(4,1)=1.720, p>0.05, respectively for 5.6 mg/mL and vehicle, as shown in FIG. 3B.

PREG at the 11.2 mg/mL dose showed a significant effect of intranasal pregnenolone administration on acetylcholine (ACh) release in the amygdala. There was a significant effect between subjects for drug, F (14,2)=4.281, p=0.035, partial eta squared=0.379. Post hoc test with multiple comparisons Dunnett 2-sided between the pregnenolone (PREG) 11.2 mg/mL drug dose and vehicle showed M=101.51 and SE=37.42, p=0.031. One-way ANOVA for the different time points was performed to further analyze the differences between PREG 11.2 mg/mL and vehicle. There were significant differences between PREG 11.2 mg/mL and vehicle at the 40, 50, 60, 70, 80, 90 minutes after treatment as shown in table 2 below.

TABLE 2
Statistical Analysis of Results

| Time after | Difference in extracellular release | |
|---|---|---|
| administration | of acetylcholine (ipsilateral vs. | |
| (min) | contralateral hemisphere of amygdala) | |
| 40 | F(2, 16) = | 3.505, p = 0.058 |
| 50 | F(2, 16) = | 4.944, p = 0.024 |
| 60 | F(2, 16) = | 6.06, p = 0.013 |
| 70 | F(2, 16) = | 8.529, p = 0.004 |
| 80 | F(2, 16) = | 3.9, p = 0.045 |
| 90 | F(2, 16) = | 4.992, p = 0.023 |

Further post hoc test Dunnett 2-sided, showed significant difference between PREG 11.2 mg/mL and vehicle at the 40, 50, 60, 70, 80, 90 minutes after treatment as shown in table 3 below.

TABLE 3
Statistical Analysis of Results (post hoc Dunnett 2-sided test)

| | Difference in extracellular release |
|---|---|
| Time after | of acetylcholine (ipsilateral vs. |
| administration | contralateral hemisphere of amygdala) |
| 40 | M = 202.78, SE = 80.27, p = 0.045 |
| 50 | M = 221.996, SE = 75.58, p = 0.02 |
| 60 | M = 196.47, SE = 59.01, p = 0.013 |
| 70 | M = 125.21, SE = 32.22, p = 0.003 |
| 80 | M = 169.62, SE = 65.94, p = 0.041 |
| 90 | M = 141.44, SE = 47.54, p = 0.019 |

Thus, PREG 11.2 mg/mL induced an increase in acetylcholine release in the amygdala, as shown in FIG. 3C.

1. A method of ipsilaterally increasing acetylcholine activity in brain tissue of a non-rodent subject in need thereof, comprising intranasally administering to the non-rodent subject a pregnenolone formulation, wherein the pregnenolone formulation is a pharmaceutical composition adapted for intranasal administration comprising an effective amount of pregnenolone in a pharmaceutically acceptable carrier.

2. The method of claim 1, wherein the pregnenolone formulation is administered only to one nostril, and acetylcholine activity is increased in an ipsilateral brain hemisphere of said nostril.

3. The method of claim 2, wherein acetylcholine activity is not substantially increased in a contralateral brain hemisphere of said nostril.

4. The method of claim 1, wherein the method results in increased acetylcholine activity in amygdala of the subject.

5. The method of claim 1, wherein the method results in increased acetylcholine activity in hippocampus of the subject.

6. The method according to claim 1, wherein the acetylcholine activity is increased within 10 minutes.

7. The method according to claim 1, wherein acetylcholine activity in the brain tissue is sustained for at least 60 minutes.

8. The method according to claim 1, wherein acetylcholine activity in the brain tissue is sustained for at least 100 minutes.

9. The method according to claim 1, wherein the effective amount of pregnenolone is from about 0.01 mg to about 2.0 mg per kilogram of bodyweight of the subject.

10. The method according to claim 1, wherein the pharmaceutically acceptable carrier comprises (a) at least one lipophilic or partly lipophilic carrier present in an amount of from about 60% to about 98% by weight of the formulation; (b) at least one compound having surface tension decreasing activity present in an amount of from about 1% to about 20% by weight of the formulation; and (c) at least one viscosity regulating agent present in an amount of from about 0.5% to about 10% by weight of the formulation.

11. The method according to claim 1, wherein the pregnenolone is loaded onto a surface of a porous excipient located inside pores of the porous excipient.

12. The method according to claim 1, wherein the subject is a human, a non-human primate, a dog, a cat, a cow, a sheep, a horse, or a rabbit.

13. The method according to claim 1, wherein the subject is suffering from a disease or condition associated with decreased acetylcholine activity in the brain.

14. The method of claim 13, wherein the disease or condition is selected from schizophrenia, Parkinson's disease, Alzheimer's disease, Lewy Body Dementia, apathy, autism, anxiety, stress, rheumatoid arthritis, traumatic brain injury, stroke, poststroke neuroprotection, bipolar disorder, depression, attention deficit hyperactivity disorder, and sleep disorders.

15. The method according to claim 1, wherein the method is effective to improve cognitive function such as memory and learning deficits.

16-33. (canceled)
